(19) United States
(12) Patent Application Publication
(10) Pub. No.: US 2004/0086912 A1
(43) Pub. Date: May 6, 2004

(54) Method for detecting foreign DNA in a host Genome

(76) Inventors: Shujun Luo, Castro Valley, CA (US); Martin Kuiper, Kessel-Lo (BE); Jen-I Mao, Palo Alto, CA (US); Glenn Albrecht, Palo Alto, CA (US); Marc Cornelissen, Lovendegem (BE)

Correspondence Address: PERKINS COIE LLP, P.O. BOX 2168, MENLO PARK, CA, 94026, US

(21) Appl. No.: 10/606,400

(22) Filed: Jun. 20, 2003

Related U.S. Application Data
(60) Provisional application No. 60/390,563, filed on Jun. 21, 2002.

(51) Int. Cl. C12Q 1/68

(52) U.S. Cl. 435/6

(57) ABSTRACT

Methods for detecting the presence of foreign DNA in the DNA of a host organism are described. Probe libraries prepared from modified DNA, and from unmodified DNA, are competitively hybridized with reference DNA sequences cloned on solid phase supports, where the reference DNA sequences are characteristic of the foreign DNA. Probes containing the foreign DNA can thereby by isolated and/or identified.

This application claims priority to U.S. Provisional Application Serial No. 60/390,563, filed Jun. 21, 2002, which is hereby incorporated by reference in its entirety.

### FIELD OF THE INVENTION

The invention relates to methods for detecting the presence of foreign DNA in the DNA of a host organism. The methods include competitive hybridization of probe libraries prepared from host DNA suspected to contain the foreign DNA, and from the native host DNA, with reference DNA sequences cloned on solid phase supports, where the reference DNA sequences are characteristic of the foreign DNA, to detect and/or isolate probes containing the foreign DNA.

### BACKGROUND

Genetic modification of crop plants using recombinant DNA technology has been widely used to confer desired properties, such as resistance to disease and herbicides, to the plants. Transformation strategies employ introduction of expression vectors into explant cells, using, for example, the natural gene transfer system of Agrobacterium tumefaciens (H Klee et al., Ann. Rev. Plant Physiol. 38:467-486, 1987; A N Binns, Physiol. Plant. 79:135-139, 1990), or physical methods such as microinjection, electroporation (M Fromm et al., Methods Enzymol. 153:351-366, 1987; C A Rhodes et al., Science 240:204-207, 1988), or microparticle bombardment, also known as “biolistics” (T M Klein et al., Proc. Natl. Acad. Sci USA 85:9502-8505, 1988; E M Southgate et al., Biotechnol. Adv. 13(4):631-651, 1995; H Daniell, Methods Mol. Biol. 62:463-489, 1997), which relies on the bombardment of target cells or tissues with microparticles which are coated with DNA. Bacterial cells themselves have been used as projectiles (J L Rasmussen et al., Plant Cell Reports 13 (3-4):212-217, 1994); generally, however, the microparticles are coated with plasmid DNA.

It has been noted in some instances of genetic modification that fragments of DNA from the non-host moieties employed for transformation can become incorporated into the DNA of the transformant. For example, backbone DNA of Agrobacterium tumefaciens Ti plasmid (about 300 kbases), exclusive of the T-DNA and transformant DNA, may be incorporated into the host. DNA of a host cell used for replication, such as E. coli, may also be incorporated, particularly in biolistic procedures. Therefore, it is desirable to be able to detect such contamination. Because the degree of homology between the DNA of the transformant and the contaminating (non-host) DNA is often high, detection of the non-host DNA by conventional hybridization to an array can be plagued by high levels of cross-hybridization. In addition, the size of the source of possible contaminating DNA is often much greater than the size of probes used in conventional hybridization detection; e.g. ˜1.5 kilobase probes in comparison to the ˜300 kilobase backbone of Ti plasmid, or the much larger E. coli genome. Therefore, several hundred probes and hybridizations would be required for complete (preferably twofold) coverage of possible contaminating DNA.

Accordingly, it would be useful to provide an efficient method for screening transformed plant strains for such contamination. Such a screening method could be used, more generally, for detection of any type of foreign DNA in a modified host genome. Examples include detection of the DNA insert itself in genetically modified organisms, or detection of a virus or other pathogen infecting a host species.

### SUMMARY OF THE INVENTION

In one aspect, the invention provides a method for detecting foreign DNA in a modified host genome, where the foreign DNA comprises DNA from a non-host or foreign genome, which is not present in the unmodified host genome. The method comprises the steps of:

a) competitively hybridizing first and second populations of polynucleotide probes with a reference DNA population, the reference DNA population comprising DNA sequences characteristic of said foreign DNA, wherein different DNA sequences are attached to separate solid phase supports in clonal subpopulations;

wherein each of the first population of polynucleotide probes comprises a DNA fragment from the unmodified host genome, and has a first label; and each of the second population of polynucleotide probes comprises a DNA fragment from the modified host genome, and has a second, distinguishable label;

thereby forming duplexes between the DNA sequences of the reference DNA population and the polynucleotide probes; and

b) sorting the solid phase supports, according to the ratio of the first label to the second label on the duplexed probes hybridized to each support.

In the formation of duplexes in step (a) the probes from the respective population are present in duplexes on each solid phase support in a ratio directly related to the relative abundance of the corresponding reference DNA sequence (or portion of the reference DNA sequence to which the probe is hybridized) in the modified genome as compared to the host genome

Subsequently, solid phase supports having a ratio of fluorescent signals which falls within a selected range of values different from 1:1 are selected, and the attached sequences or, preferably, the hybridized probes on the selected solid phase supports are identified, typically by sequencing some portion of the hybridized probes.

The steps of the method may also comprise, prior to the competitive hybridization:

(i) providing the reference DNA population, comprising DNA sequences characteristic of the foreign DNA, wherein different sequences are attached to separate solid phase supports in clonal subpopulations;

(ii) providing the first population of polynucleotide probes, each comprising a DNA fragment from the unmodified host genome, not containing the foreign DNA, and having a first label; and

(iii) providing the second population of polynucleotide probes, each comprising a DNA fragment from the modified host genome, suspected to contain the foreign DNA, and having a second, distinguishable label.

In a preferred embodiment of the method, the first and second labels on the probe populations are first and second distinguishable fluorescent labels. In another preferred embodiment, the solid phase supports are microparticles, and the microparticles are sorted in step (e) by FACS, according to the ratio of fluorescent signals generated by the fluorescent labels on each microparticle.

In one embodiment, the probes derived from the host genome and those from the modified genome have a length such that some portion of the probes not containing the foreign DNA are able to hybridize with the reference DNA sequences, under the conditions of hybridization employed in the assay.

The first and second probe populations can be prepared by: preparing a restriction digest or sheared fragments from the host genome or the modified genome, ligating pairs of PCR adapters to the fragments, and amplifying the fragments by PCR, using primers hybridizing to the adapter sequences. Preferably, each pair of PCR primers includes at least one labeled primer. Typically, the primers are fluorescently labeled, and primers used to amplify probes from the host genome have labels distinguishable from those on primers used to amplify probes from the modified genome.

In one embodiment of the method, multiple rounds of competitive hybridization are carried out; e.g.: each probe population is separately hybridized with the reference DNA population, and then recovered from the solid phase supports; each recovered probe population is then amplified by PCR, preferably using primers labeled with first and second distinguishable labels, respectively, and used for competitive hybridization.

An exemplary non-host genome, or source of foreign DNA, is E. coli, where the modified genome is that of a transgenic plant modified with DNA which was replicated in E. coli. The transgenic plant is, for example, a genetically modified crop plant. As noted above, the probes derived from the host genome and those from the modified genome preferably have a length such that some portion of the probes not containing the foreign DNA are able to hybridize with the reference DNA sequences, under the conditions of hybridization employed in the assay. In the case of E. coli and a transgenic plant as described above, the probe sequences are preferably at least 100 nucleotides in length, up to about 1.5 kilobases in length.

The method is especially useful for analyses in which there is a high degree of homology between the DNA of the host organism and the foreign or non-host organism, and/or the total size of the source of the non-host DNA to be detected is much greater than the size of the hybridization probes. In such cases, detection of the foreign DNA by conventional hybridization to an array can be laborious and may exhibit high levels of cross-hybridization.

The method of the invention can be used, as described above, to detect contaminant DNA of a non-host organism used to replicate insert DNA, in GM (genetically modified) organisms made by direct gene transfer, or to detect extraneous incorporation of Ti-plasmid sequences outside of the T-DNA insert, in GM plants made by Agrobacterium mediated transfer.

The method can further be used, in general, for any detection of foreign DNA in a host, such as in detection of different possible (i.e. commercially approved or existing) transgenes in a batch of plant material. The method can also be used to detect viral or other pathogenic infection in an organism.

The method can be used for screening a plurality of transgenic organisms for one or more types of foreign DNA. In this aspect, the modified host genome is one of a plurality of transgenic organisms, such as transgenic plant lines, and the method further comprises:

carrying out step (i) (i.e. providing a reference DNA population) for each type of foreign DNA suspected of being present in the plurality of transgenic organisms;

carrying out step (ii) (i.e. providing a first population of polynucleotide probes) for each different type of unmodified host genome represented in the plurality;

carrying out step (iii) (i.e. providing a second population of polynucleotide probes) for each transgenic organism of the plurality;

carrying out steps (a)-(d) for each transgenic organism of the plurality, and for each type of foreign DNA suspected of being present in that organism, to determine the presence or absence of foreign DNA in each organism of the plurality; and

selecting one or more of the plurality of transgenic organisms, according to a predetermined selection criterion based on the presence of the foreign DNA in the organism.

In a related aspect, the invention provides a kit for use in detecting foreign DNA in a modified host genome, the foreign DNA comprising DNA from a non-host or foreign genome; the kit comprising:

(i) a reference nucleic acid library containing genomic DNA sequences from the foreign genome, wherein different sequences are attached to separate solid phase supports in clonal subpopulations;

(ii) a first plurality of probes, derived from a nucleic acid library from the host genome, not including the foreign DNA, and having a first label, and

(iii) a second plurality of labeled probes, derived from a nucleic acid library from the modified host genome suspected to contain the foreign DNA, and having a second label distinguishable from the first label. Preferably, the labels comprise fluorescent compounds.

These and other objects and features of the invention will become more fully apparent when the following detailed description of the invention is read in conjunction with the accompanying drawings.

### BRIEF DESCRIPTION OF THE DRAWINGS

FIGS. 1A-B illustrate one method for preparation of a nucleic acid reference library, which includes the steps of: inserting DNA fragments into a tag-containing vector, amplifying tag-DNA conjugates out of the vector, loading the amplified conjugates onto microparticles, isolating DNA-loaded microparticles, and preparing the reference library for competitive hybridization;

FIG. 2 illustrates optimization of probe libraries, including the use of a first round of hybridization for reducing complexity of probe populations, and the use of UDG (uracyl DNA glycosylase) for digestion of reference DNA sequences, prior to PCR amplification of hybridized probe sequences, in accordance with preferred embodiments of the invention;

FIG. 3 illustrates competitive hybridization of the probe populations with a reference DNA library cloned on solid phase supports, in accordance with one embodiment of the invention;

FIGS. 4A-E illustrate FACS analysis of a bead-supported reference library which has been hybridized (separately) with two probe libraries, showing two rounds of hybridization, for reducing complexity of the probe populations: A=control beads; B=1^{st} hybridization with PHI1 probes; C=1^{st} hybridization with GU262 probes; D=2nd hybridization with PHI1 probes, labeled; E=2^{nd} hybridization with GU262 probes, labeled;

FIGS. 5A-B illustrate procedures that may be used for sequencing DNAs isolated by FACS sorting; and

FIG. 6 illustrates FACS analysis of microparticles loaded with probe DNA strands labeled with two different fluorescent dyes, resulting from competitive hybridization of PHI1 (native) and GU262 (transgenic) probe DNA with a microparticle-supported E. coli DNA reference library, in accordance with one embodiment of the invention.

### DEFINITIONS

The terms below have the following meanings unless indicated otherwise.

A “modified host organism”, as used herein, refers to an organism into which exogenous DNA has been introduced or is suspected to have been introduced. The term “modified host genome” is employed in a similar manner.

The exogenous DNA, also referred to as “foreign DNA” or “non-host DNA”, is DNA not occurring in the genome of the unmodified host organism. Typically, it refers to DNA whose presence is sought to be detected by the method of the invention.

The “unmodified host genome” refers to the genome of the host organism which is known not to include the foreign DNA whose presence is sought to be detected. It may also be referred to as the “unmodified host genome” or “unmodified DNA” of the host organism, or simply as the host genome.

DNA sequences “characteristic of” the foreign DNA refer to a representative population of sequences from the foreign DNA suspected of occurring in a modified genome, and desired to be detected if present. Such a population is typically a restriction and/or sheared digest of the foreign DNA.

A “complement” or “tag complement”, as used herein in reference to oligonucleotide tags, refers to an oligonucleotide to which a oligonucleotide tag specifically hybridizes to form a perfectly matched duplex or triplex. In embodiments where specific hybridization results in a triplex, the oligonucleotide tag may be selected to be either double stranded or single stranded. Thus, where triplexes are formed, the term “complement” is meant to encompass either a double stranded complement of a single stranded oligonucleotide tag or a single stranded complement of a double stranded oligonucleotide tag.

The term “oligonucleotide” as used herein includes linear oligomers of natural or modified monomers or linkages, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. When an oligonucleotide is represented by a sequence of letters, such as “ATGCCTG,” it will be understood that the nucleotides are in 5′→3′ order from left to right, and that “A” denotes deoxyadenosine, “C” denotes deoxycytidine, “G” denotes deoxyguanosine, and “T” denotes thymidine, unless otherwise noted. Usually, oligonucleotides comprise the four natural nucleotides; however, they may also comprise non-natural nucleotide analogs. It is clear to those skilled in the art when oligonucleotides having natural or non-natural nucleotides may be employed; e.g., where processing by enzymes is called for, usually oligonucleotides consisting of natural nucleotides are required.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

As used herein, “nucleoside” includes the natural nucleosides, including 2′-deoxy and 2′-hydroxyl forms, e.g. as described in Kornberg and Baker, DNA Replication, 2nd Ed. (Freeman), San Francisco, 1992. “Analogs”, in reference to nucleosides, includes synthetic nucleosides having modified base moieties and/or modified sugar moieties, e.g. as described by Scheit, Nucleotide Analogs (John Wiley, New York, 1980); Uhlman and Peyman, Chemical Reviews 90: 543-584 (1990), or the like, with the proviso that they are capable of specific hybridization. Such analogs include synthetic nucleosides designed to enhance binding properties, reduce complexity, increase specificity, and the like.

As used herein, “sequence determination” or “determining a nucleotide sequence”, in reference to polynucleotides, includes determination of partial as well as full sequence information of the polynucleotide. That is, the term includes sequence comparisons, fingerprinting, and like levels of information about a target polynucleotide, as well as the express identification and ordering of nucleosides, usually each nucleoside, in a target polynucleotide. The term also includes the determination of the identification, ordering, and locations of one, two, or three of the four types of nucleotides within a target polynucleotide. For example, in some embodiments sequence determination may be effected by identifying the ordering and locations of a single type of nucleotide, e.g. cytosines, within the target polynucleotide “CATCGC . . .” so that its sequence is represented as a binary code, e.g. “100101 . . . ” for “C—(not C)—(not C)—C—(not C)—C . . . ” and the like.

As used herein, the term “complexity” in reference to a population of polynucleotides refers to the number of different species of polynucleotide present in the population.

### DETAILED DESCRIPTION OF THE INVENTION

I. Introduction

Co-owned U.S. Pat. No. 6,265,163, which is incorporated herein by reference, provides a method of massive parallel analysis of all or a substantial fraction of expressed genes, allowing selection of differentially expressed genes from non-differentially expressed genes, without requiring prior knowledge of the differentially expressed sequences being monitored. In accordance with that method, differently labeled populations of DNAs from sources to be compared are competitively hybridized with reference DNA cloned on solid phase supports, e.g. microparticles, to provide a differential expression library which, in the preferred embodiment, is manipulated by fluorescence-activated cell sorting (FACS). Monitoring the relative signal intensity of the different fluorescent labels on the microparticles permits quantitative analysis of relative expression levels between the different sources.

Labeled DNA or RNA probes from the samples to be compared are competitively hybridized to the DNA sequences of the reference DNA population using conventional hybridization conditions, e.g. such as disclosed in Schena et al., Science 270: 467-469 (1995), and DeRisi et al., Science 278: 680-686 (1997). After hybridization, an optical signal is generated by each of the two labeled species of DNA or RNA probes, so that a relative optical signal is determined for each microparticle. The optical signal is preferably generated by a fluorescent label directly attached to the probe. Such optical signals are measured in a fluorescence activated cell sorter, or like instrument, which permits the microparticles whose relative optical signal fall within a predetermined range of values to be sorted and accumulated.

Populations of microparticles having relative signal intensities of interest are isolated by FACS, and the attached DNAs may then be identified by sequencing, such as with massively parallel signature sequencing (MPSS) (see Brenner, U.S. Pat. No. 5,695,934, or Albrecht et al., U.S. Pat. No. 6,013,445, both of which are incorporated herein by reference), or with conventional DNA sequencing protocols. Frequently, only a portion of the DNAs need be sequenced for identification purposes.

An illustration of the process is also provided in Brenner el al., PNAS 97:1665-70 (2000).

Such methods also can be used for identifying differentially represented variations in genomic DNA, e.g. SNP's, deletions, or duplications. An important benefit of these methods is that the identity of the differentially represented DNA sequences being detected need not be known prior to analysis.

In the methods described in the above-cited documents, the composition of the reference DNA is usually substantially similar to that of the probe DNA, with the exception of differentially expressed sequences, in the case of cDNA, or in polymorphic variations such as SNP's, deletions, and mutations, in the case of genomic DNA. For example, when these methods are used for analysis of differentially regulated or expressed genes, the probe populations are cDNA libraries derived from expressed genes of each of a plurality of sources selected from different cells, tissues, or individuals, and the reference DNA library is derived from genes expressed in the plurality of different sources. When the methods are used for analysis of genetic variations among individuals, the probe populations are genomic DNA libraries derived from different individuals, and the reference DNA library is typically derived from pooled genomic DNA of such individuals.

It has been found, and is the subject of the present invention, that such competitive hybridization of labeled probes to a reference library can also be used to detect foreign or contaminant DNA within a modified organism, such as a transgenic organism, in comparison to the DNA of the unmodified organism (that is, the organism not having the modification being detected). The probe populations are derived from the modified organism and the unmodified organism, respectively. In the methods described herein, the reference DNA library is derived, not from these sources, but from the foreign DNA which is the source of the expected contamination.

Preferably, the probe sequences are of sufficient length such that some portion of the probes which do not contain the foreign DNA are able to hybridize, at a given level of stringency, with the reference DNA library. The competitive hybridization is carried out at or below such a level of stringency. For example, the process can be used to detect contaminating E. coli sequences in a plant, such as a crop plant, which has been transgenically modified using a vector replicated in E. coli. The genomes of many such crop plants include sequences having a substantial degree of homology with the E. coli genome, which can complicate conventional detection by hybridization to a fixed array of sequences. In an example described below, the present process was used to detect E. Coli contamination in a strain of soybean transgenically modified for herbicide resistance.

The following sections will describe in more detail procedures used in carrying out various embodiments of the invention.

II. Oligonucleotide Tags for Solid Phase Cloning of Polynucleotides

Oligonucleotide “tags” are preferably used to construct reference DNA populations attached to solid phase supports, preferably microparticles, for use in the method of the invention. Such tags and methods of their preparation and use are described in detail in PCT Pubn. Nos. WO 96/41001 and WO 96/12014 and in co-owned U.S. Pat. No. 5,604,097, which are incorporated herein by reference in their entirety. Oligonucleotide tags, when used with their corresponding tag complements, provide a means of enhancing specificity of hybridization for sorting, tracking, or labeling molecules, especially polynucleotides, such as cDNAs or mRNAs derived from expressed genes.

Oligonucleotide tags for sorting may range in length from 12 to 60 nucleotides or basepairs. Preferably, oligonucleotide tags range in length from 18 to 40 nucleotides or basepairs, and more preferably from 25 to 40 nucleotides or basepairs. Preferably, repertoires of single stranded oligonucleotide tags for sorting contain at least 100 members; more preferably, repertoires of such tags contain at least 1000 members; and most preferably, repertoires of such tags contain at least 10,000 members. As used herein in reference to oligonucleotide tags and tag complements, the term “repertoire” means the total number of different oligonucleotide tags or tag complements that are employed for solid phase cloning (sorting) or for identification.

Preferably, tag complements in mixtures, whether synthesized combinatorially or individually, are selected to have similar duplex or triplex stabilities to one another so that perfectly matched hybrids have similar or substantially identical melting temperatures. This permits mismatched tag complements to be more readily distinguished from perfectly matched tag complements in the hybridization steps, e.g. by washing under stringent conditions.

When oligonucleotide tags are used for sorting, as is the case for constructing a reference DNA population, tag complements are preferably attached to solid phase supports. Such tag complements can be synthesized on the surface of the solid phase support, such as a microscopic bead or a specific location on an array of synthesis locations on a single support, such that populations of identical, or substantially identical, sequences are produced in specific regions. Preferably, tag complements are synthesized combinatorially on microparticles, so that each microparticle has attached many copies of the same tag complement. A wide variety of microparticle supports may be used with the invention, including microparticles made of controlled pore glass (CPG), highly cross-linked polystyrene, acrylic copolymers, cellulose, nylon, dextran, latex, polyacrolein, and the like, as known in the art.

Polynucleotides to be sorted, or cloned onto a solid phase support, each have an oligonucleotide tag attached, such that different polynucleotides have different tags. This condition is achieved by employing a repertoire of tags substantially greater than the population of polynucleotides and by taking a sufficiently small sample of tagged polynucleotides from the full ensemble of tagged polynucleotides. After such sampling, when the populations of supports and polynucleotides are mixed under conditions which permit specific hybridization of the oligonucleotide tags with their respective complements, identical polynucleotides sort onto particular beads or regions. The sampled tag-polynucleotide conjugates are preferably amplified, e.g. by polymerase chain reaction, cloning in a plasmid, RNA transcription, or the like, to provide sufficient material for subsequent analysis.

An exemplary tag library for use in sorting is shown below (SEQ ID NO: 1).

| Formula I | |
|---|---|
| Left Primer | |
| 5′AGAATTCGGGCCTTAATTAA | (SEQ ID NO: 2) |
| 5′AGAATTCGGGCCTTAATTAA[⁴(A,G,T)₈]GGGCCC- | {SEQ ID NO:1 start} |
| TCTTAAGCCCGGAATTAATT[⁴(T,C,A)₈]CCCGGG- | |
| ↑ ↑ ↑ | |
| EcoRI PacI Bsp1201 | |
| BbsI BamHI | |
| ↓ ↓ | |
| -GCATAAGTCTTCXXX...XXXGGATCCGAGTGAT-3′ | {SEQ ID NO:1 cont.} |
| -CGTATTCAGAAGXXX...XXXCCTAGGCTCACTA | |
| Right Primer | |
| XXXXXCCTAGGXTCACTA-5′ | (SEQ ID NO: 3) |

The flanking regions of the oligonucleotide tag may be engineered to contain restriction sites, as exemplified above, for convenient insertion into and excision from cloning vectors. Optionally, the right or left primers (SEQ ID NOs: 3 and 2, respectively) may be synthesized with a biotin attached (using conventional reagents, e.g. available from Clontech Laboratories, Palo Alto, Calif.) to facilitate purification after amplification and/or cleavage. Preferably, for making tag-fragment conjugates, the above library is inserted into a conventional cloning vector, such as pUC19, or the like. Optionally, the vector containing the tag library may contain a “stuffer” region, “XXX . . . XXX,” which facilitates isolation of fragments fully digested with, for example, BamHI and BbsI.

Sorting and attachment of populations of DNA sequences in a reference library, e.g. a cDNA or genomic library, to microparticles or to separate regions on a solid phase support is carried out such that each microparticle or region has substantially only one kind of sequence attached; that is, such that the DNA sequences are present in clonal subpopulations. Preferably, at least ninety-five percent of the DNA sequences have unique tags attached. This objective is accomplished by ensuring that substantially all different DNA sequences have different tags attached. This condition, in turn, is brought about by sampling the full ensemble of tag-DNA sequence conjugates for analysis. (It is acceptable that identical DNA sequences have different tags, as it merely results in the same DNA sequence being operated on or analyzed twice.) Such sampling can be carried out either overtly—for example, by taking a small volume from a larger mixture—after the tags have been attached to the DNA sequences; it can be carried out inherently as a secondary effect of the techniques used to process the DNA sequences and tags; or sampling can be carried out both overtly and as an inherent part of processing steps. If a sample of n tag-DNA sequence conjugates are randomly drawn from a reaction mixture, as could be effected by taking a sample volume, the probability of drawing conjugates having the same tag is described by the Poisson distribution, P(r)=e^{-λ}(λ)^{r}/r, where r is the number of conjugates having the same tag and λ=np, where p is the probability of a given tag being selected. If n=10⁶ and p=1/(1.67×10⁷) (for example, if eight 4-base words as described in Brenner et al. were employed as tags), then λ=0.0149 and P(2)=1.13×10⁻⁴. Thus, a sample of one million molecules gives rise to an expected number of doubles well within the preferred range. Such a sample is readily obtained by serial dilutions of a mixture containing tag-fragment conjugates.

Preferably, DNA sequences are conjugated to oligonucleotide tags by inserting the sequences into a conventional cloning vector carrying a tag library. For example, DNA fragments may be constructed having a Bspl20I site at their 5′ ends and, after digestion with Bspl20I and another enzyme such as Sau3A or DpnII, may be directionally inserted into a pUC 19 vector carrying the tags of Formula I, to form a tag-DNA library, which includes every possible tag-DNA pairing. A sample is taken from this library for amplification. Sampling may be accomplished by serial dilutions of the library, or by simply picking plasmid-containing bacterial hosts from colonies.

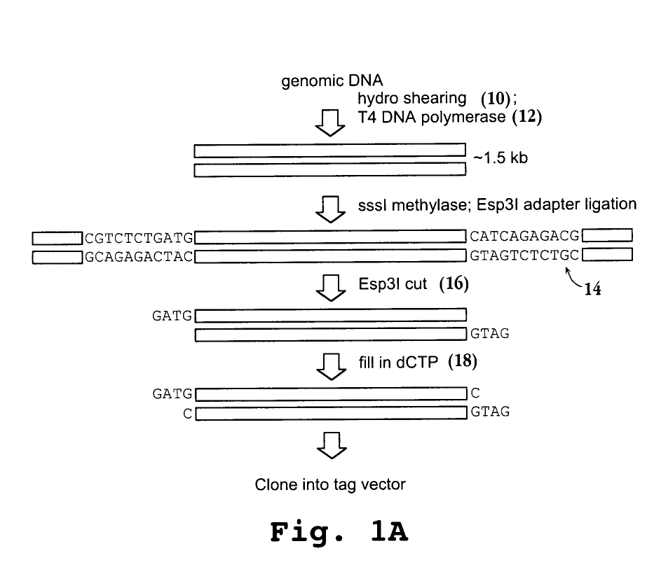

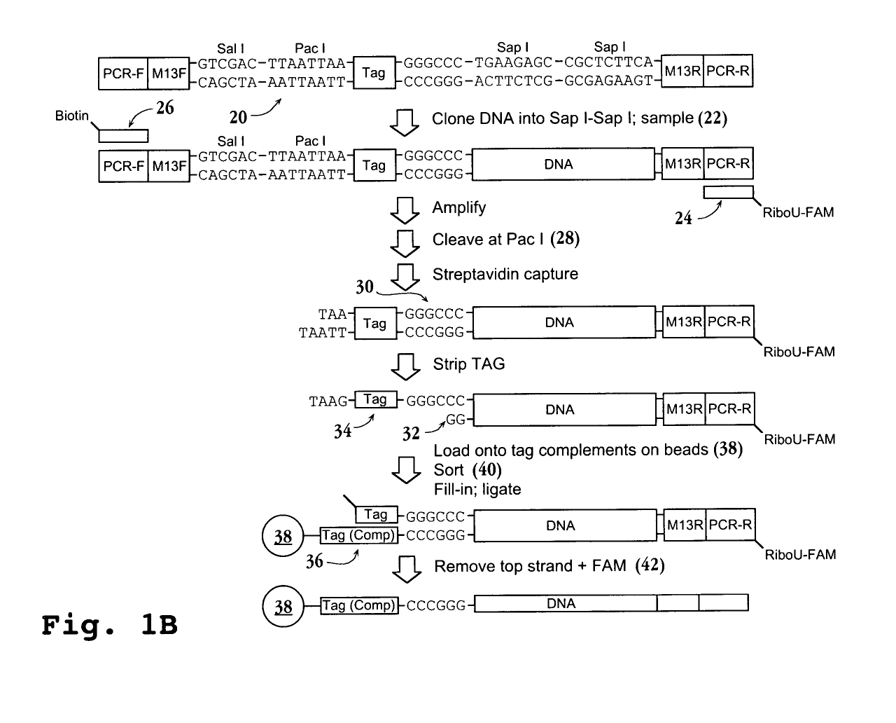

The tag-DNA conjugates are mixed with microparticles containing the tag complements (e.g. as shown in FIG. 1B, discussed further below) under conditions that favor the formation of perfectly matched duplexes between the tags and their complements. There is extensive guidance in the literature for creating these conditions. Exemplary references providing such guidance include Wetmur, Critical Reviews in Biochemistry and Molecular Biology, 26: 227-259 (1991); Sambrook et al., Molecular Cloning: A Laboratory Manual, 2nd Edition (Cold Spring Harbor Laboratory, New York, 1989); and the like. Preferably, the hybridization conditions are sufficiently stringent so that only perfectly matched sequences form stable duplexes. Finally, the microparticles are washed to remove polynucleotides with mismatched tags.

III. Preparation of Reference Libraries

A reference DNA population may consist of any set of DNA sequences which includes sequences whose occurrence in a test population is sought to be determined. In one embodiment, the reference DNA population is prepared from genomic DNA of the organism which is the source of the foreign DNA being detected.

Once the DNA sequences making up a reference DNA population are obtained, they are preferably attached to discrete solid surfaces, e.g. separated microbeads or discrete regions of a planar array, as described further below. In one embodiment, these reference DNA sequences are conjugated with oligonucleotide tags for such solid phase cloning. Preferably, the DNA sequences are prepared so that they can be inserted into a vector carrying an appropriate tag repertoire, as described above, to form a library of tag-DNA sequence conjugates. A sample of conjugates is taken from this library, amplified, and loaded onto microparticles, as described further below. See also co-owned U.S. Pat. Nos. 5,604,097, 6,265,163, and 6,235,475.

One procedure for preparing a reference DNA population of nucleic acid fragments as clonal subpopulations on solid particle supports is as follows (see FIGS. 1A-1B). Genomic DNA, in this case E. coli DNA, is sheared (10) to produce large fragments, e.g. about 1.5-2 kb, which are then treated with T4 polymerase (12) to produce blunt ends. The fragments are methylated with SSSI methylase, and ligated to a blunt-end Esp3I adaptor (14). One example of such an adaptor is as follows:

| | |
|---|---|
| 5′-CTTCGTACCGACCGTCTCTGATG-3′ | |
| 3′-CGAAGCATGGCTOGCAGAGACTAC_{P}-5′ | (SEQ ID NO: 4) |

With continued reference to FIG. 1A, the fragments are then cut with Esp3I (16), and the cleaved ends are filled in with dCTP (18), producing a 3-base overhang.

The fragments are then cloned into a tag-conjugate vector library (20, FIG. 1B) prepared from a tag vector such as the MS SI tag vector below (SEQ ID NO: 5). The vector library is prepared by cloning oligonucleotide tags, as described above, into a cloning site, such as the BseRI-Bspl20I site of the vector shown as SEQ ID NO: 5.

| | |
|---|---|
| EcoRI PCR-F-------------------> | (SEQ ID NO: 5) |
| GAATTCTGAATAAATAGCGCCAGGGTTTTCCCAGTCACGACG- | |
| M13F------------->SalI | |
| TGTAAAACGACGGCCAGTCGACCGTCCAGACTTCTACTACCTCAC- | |
| PacI Bsp120I | |
| TTAATTAAGGAATAGGCCTCTCCTCGAGCTCGGTACCGGGCCC- | |
| BseRI | |
| GCTTCACAGATGTCGGCTAATGCATAAGTCTTCATCTGCAGATT - | |
| GAAGAGCGATATCGCTCTTCAATCGGATGCTGACAAGATACGACCACGCGGCCGC- | |
| SapI SapI | |
| GGTCATAGCTGTTTCCTGCCACACAACATACGAGCCGGAAGC- | |
| <-------------M13R<------------------PCR-R | |
| TCAACTAATTAAGCTT | |
| HindIII | |

The genomic DNA fragments are cloned into the SapI-SapI site of the vector (22).

A sample is then taken of the vectors containing tag-DNA conjugates, as described in Section II above. Preferably, the sample is large enough so that there is a high probability that all of the different types of DNA sequences will be represented on the loaded microparticles.

After the tag-DNA sequence conjugates are sampled, they can be amplified by PCR using a fluorescently labeled primer (e.g. PCR-R-riboU-FAM, 24), to provide sufficient material to load onto the tag complements of the microparticles. The fluorescent label provides a means for distinguishing loaded from unloaded microparticles, as disclosed in Brenner et al., U.S. Pat. No. 5,604,097. Accordingly, the tag-DNA conjugates are amplified from the vector by PCR, using biotinylated primer (PCR-F-biotin, 26) and labeled primer (PCR-R-riboU-FAM, 24), in the presence of 5-methyldeoxycytidine triphosphate. The resulting amplicon is isolated by streptavidin capture, as indicated in FIG. 1B.

In one embodiment, the riboU-FAM primer has the sequence:

5′-FAM-spacer-spacer-UGCTTCCGGCTCGTATGTTGTGTGG-3′ (SEQ ID NO: 6) The ribophosphate diester bond at the riboU of the primer can be later cleaved by selective specific base hydrolysis or RNase treatment, thus excising any upstream regions, including the label, from the region downstream from the riboU site.

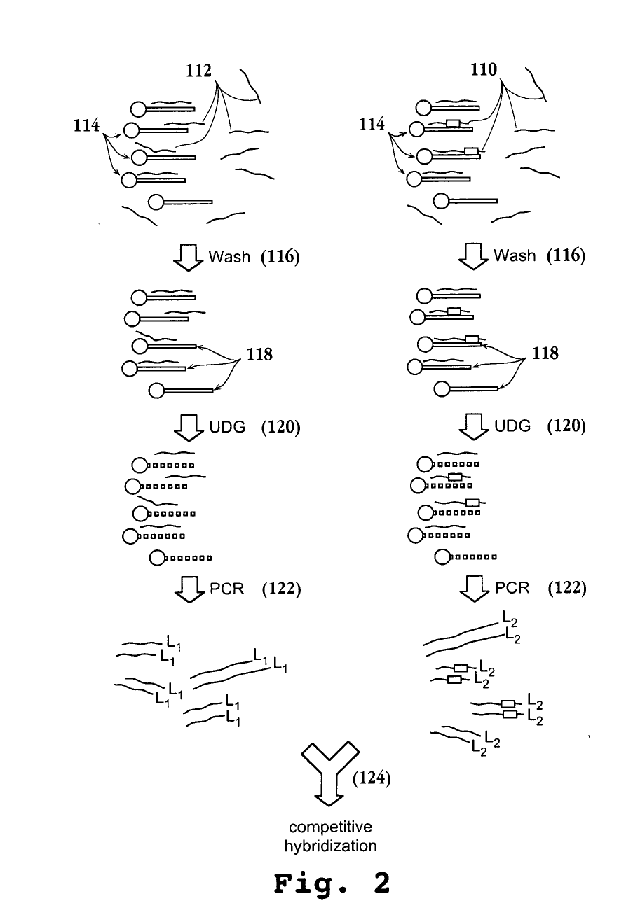

In a useful modification of the reference library preparation, PCR amplification of the tag-reference DNA conjugate library is carried out using dUTP in place of dTTP. Accordingly, the reference DNA strand loaded onto the bead (as illustrated at the bottom of FIG. 1B) has dU in place of dT. This allows the bead-bound reference strand to later be digested with uracyl DNA glycosylase. As discussed further below, and illustrated in FIG. 2, this digestion is carried out after a first round of hybridization of probes to the reference strands.

With continued reference to FIG. 1B, the vector is cleaved at a PacI site (28) to release the tag-DNA constructs from the beads. The restriction site used for release of the fragments can vary, but preferably it corresponds to a rare-cutting restriction endonuclease, such as PacI, NotI, FseI, PmeI, SwaI, or the like, which permits the captured amplicon to be released with minimal probability of cleavage occurring at a site internal to the DNA of the amplicon.

The vector is then “stripped” to render the oligonucleotide tags single-stranded, as indicated in FIG. 1B. The 3′→5′ exonuclease activity of a DNA polymerase, preferably T4 DNA polymerase, can be used for this purpose (see Brenner, U.S. Pat. No. 5,604,097). In a preferred embodiment, tags consist of subunits or “words” that contain only three of the four natural nucleotides, which can be preferentially digested from the tag-DNA conjugate in the 3′→5′ direction with the 3′→5′ exonuclease activity of a DNA polymerase. In one embodiment, the tags are designed to contain only A's, G's, and T's; thus, tag complements consist of only A′s, C′s, and T′s. When the tag-DNA conjugates are treated with T4 DNA polymerase in the presence of dGTP, the complementary strands of the tags are “stripped” away to the first G. At that point, the incorporation of dG by the DNA polymerase balances the exonuclease activity of the DNA polymerase, effectively halting the “stripping” reaction. A sequence such as 5′-GGCCC-3′ (30) adjacent the tag causes the DNA polymerase “stripping” or “chewback” reaction to be halted at the G triplet when this DNA polymerase is used with dGTP.

One of ordinary skill could make many alternative design choices for carrying out the same objective, i.e. rendering the tags single stranded. Such choices could include selection of different enzymes, different compositions of words making up the tags, and the like.

When the “stripping” or “chewback” reaction is quenched, the result is duplex 32 (FIG. 1B) with single stranded tag 34. After isolation, the tag-DNA conjugates are hybridized to tag complements 36 attached to microparticles 38.

The tag-DNA conjugates are preferably hybridized to the full repertoire of tag complements. That is, among the population of microparticles 38, there are microparticles having every tag complement sequence 36 of the entire repertoire. Thus, the tag-DNA conjugates will generally hybridize to tag complements on only about one percent of the microparticles. Loaded microparticles are separated from unloaded microparticles for further processing, as noted above, preferably by sorting (40) with a fluorescence-activated cell sorter (FACS). In the procedure illustrated in FIG. 1A-B, a fluorescent label, e.g. FAM, is attached by way of primer PCR-R-riboU-FAM.

After FACS, or like sorting, loaded microparticles are isolated, and a fill-in reaction is carried out to fill any gap between the complementary strand of the tag-DNA conjugate and the 5′ end of tag complement 36 attached to microparticle 38. The complementary strand of the tag-DNA conjugate is then ligated to the 5′ end of tag complement 36. This embodiment requires that the 5′ end of the tag complement be phosphorylated, e.g. by a kinase, such as, T4 polynucleotide kinase, or the like. The fill-in reaction is preferably carried out because the “stripping” reaction does not always halt at the first G. Preferably, the fill-in reaction uses a DNA polymerase lacking 5′→3′ exonuclease activity and strand displacement activity, such as T4 DNA polymerase. Also preferably, all four dNTPs are used in the fill-in reaction, in case the “stripping” extended beyond the G triplet.

The microparticles are then treated to remove label and to melt off the noncovalently attached strand. In one embodiment, the tag-DNA conjugates can be treated with a restriction endonuclease recognizing a site adjacent to the PCR-R primer binding site (not shown in FIG. 1B), thereby removing the label carried by the bottom strand. In the embodiment of FIG. 1B, the riboU-FAM label and the top strand are both removed (42) by treatment with NaF/NaOH.

Alternative amplification methods which do not use PCR can be used, if desired, to avoid any preferential amplification of sequences during PCR. For example, plasmid DNA can be amplified in bacteria, and the tag-DNA insert removed with appropriate restriction enzymes. The sequences are labeled, for use in separated loaded from unloaded beads by FACS, by ligating a labeled adaptor.

The number of copies of a DNA in a clonal subpopulation (i.e., the loading on a microparticle) should be sufficient to permit FACS sorting of microparticles, where fluorescent signals are generated by one or more fluorescent dye molecules attached to the DNAs attached to the microparticles, as described further below. The number can be dependent on several factors, such as the density of tag complements on the solid phase supports, the size and composition of microparticle used, the duration of the hybridization reactions, the complexity of the tag repertoire, the concentration of individual tags, the tag-DNA sample size, the labeling means for generating optical signals, the particle sorting means, signal detection system, and the like. Guidance for making design choices relating to these factors is readily available in the literature on flow cytometry, fluorescence microscopy, molecular biology, hybridization technology, and related disciplines.

Typically, number of copies per microparticle can be as low as a few thousand, e.g. 3,000-5,000, when a fluorescent molecule such as fluorescein is used, and as low as several hundred, e.g. 800-8000, when a rhodamine dye, such as rhodamine 6G, is used. Preferably, each clonal subpopulation contains at least 10⁴ copies, and more preferably at least 10⁵ copies, of a DNA sequence.

IV. Preparation of Probes

As described above, the desired reference library is prepared from an appropriate reference DNA source (e.g. the source of the foreign DNA being detected) by preparing a sheared and/or restriction digest, cloning, and loading each cloned fragment onto a spatially discrete solid support, e.g. a microparticle. Probe sets can be prepared in a similar manner from sheared/restriction digests of the modified genome being analyzed (i.e. suspected to contain the foreign DNA) and the corresponding non-modified genome, respectively. Various restriction enzymes could be used in preparing the libraries and probes, in accordance with ordinary skill in the art.

As in preparation of the reference libraries, the probe DNA is sheared or cleaved with a restriction endonuclease to produce a population of fragments. The restriction endonuclease may be any restriction enzyme whose cleavage results in fragments with predictable cleaved ends. Adaptors are then ligated to the cleaved ends, in a conventional ligation reaction, to give fragment-adaptor complexes. The adaptors are double stranded oligonucleotide adaptors which contain complementary protruding strands to those of the restriction fragments, or, for ligation to sheared libraries, the adaptors may have blunt ends. They may vary widely in length and composition, but are preferably long enough to include a primer binding site for amplifying the fragment-adaptor complexes by polymerase chain reaction (PCR). Preferably, the double stranded region of the adaptor is within the range of 14 to 30 basepairs, and more preferably, within the range of 16 to 24 basepairs.

In a preferred embodiment, the 3′ recessed strands of the fragment-adaptor complexes are first extended by one nucleotide (“fill-in”) to reduce the length of the protruding strands to three nucleotides, thereby reducing self-ligation, both of the fragments and the adaptors.

The fragment-adaptor complexes are then amplified by PCR and purified. Labeled probes, preferably incorporating a light-generating label, are prepared by incorporating a labeled nucleotide, or, more preferably, by using labeled PCR primers. Many light-generating labels are known in the art, and include fluorescent, calorimetric, chemiluminescent, and electroluminescent labels. Generally, such labels produce an optical signal which may comprise an absorption frequency, an emission frequency, an intensity, a signal lifetime, or a combination of such characteristics. Preferably, fluorescent labels, such as fluorescent dye molecules, are employed. Fluorescently labeled nucleic acids are described, for example, in Haugland, Handbook of Fluorescent Probes and Research Chemicals (Molecular Probes, Inc., Eugene, 1992); Keller and Manak, DNA Probes, 2nd Edition (Stockton Press, New York, 1993).

For preparation of probes from soybean strains GU262 (modified for herbicide resistance) and PHI1 (native), used in the Examples below, the genomic DNA from each was digested with Sau3A, and fragments were filled in with dGTP and ligated with a T3-ATC adaptor. The DNA may also be sheared and treated to give blunt ends, in which case a blunt ended adaptor, such as the T3 adaptor below, is used.

T3-ATC adapter (for sau3A cut probes):

| | | |
|---|---|---|
| T3-ATC adaptor (for sau3A cut probes): | 5′-GCAATTAACCCTCACTAAAGGGAACA-3′ | (SEQ ID NO: 7) |
| | 3′-AATTGGGAGTGATTTCCCTTGTCTA-5′ | (SEQ ID NO: 8) |
| T3 adapter (for sheared probes): | 5′-GCAATTAACCCTCACTAAAGGGAACA-3′ | (SEQ ID NO: 9) |
| | 3′-AATTGGGAGTGATTTCCCTTGT-5′ | |

Reduced complexity probes may be prepared by using a pre-hybridization step prior to the competitive hybridization. This strategy is illustrated in FIG. 2, and is described further in Example 4 for a model system and in Example 5 for an E. coli reference library. In general, each probe population (i.e. the modified host DNA probe population 110 and the native host DNA probe population 112) is separately hybridized with the bead-supported reference DNA population 114, preferably at low stringency. (Only one DNA strand per bead is shown in the Figure for the sake of clarity.) Probes which do not hybridize to any of the bead-supported reference DNA strands can then be removed (116) from the probe population by washing the beads.

The hybridizing probes are then recovered from the solid phase supports. This can be accomplished by heat denaturing of the probes from the reference sequences on the supports and filtering of the supports from the probes. In a preferred embodiment, the bead-supported reference DNA sequences include dUTP in place of dTTP, as noted above in Section III. This modification allows the bead-bound reference strands (118 in FIG. 2) to be digested (120) with uracyl DNA glycosylase, leaving intact only the hybridizing probe strands, as depicted in the Figure. This process can be combined with heat denaturing, as above, if desired, and prevents contamination of the probe strands by any reference strands which may detach from the solid supports, and which may be nonspecifically amplified during PCR.

The recovered probes are amplified and labeled via PCR (122), using primers labeled with the first or second labels, respectively. Probes from the different populations can then be combined (124) for competitive hybridization, as described further below.

V. Competitive Hybridization

In applying the method of the invention, probe sequences prepared from DNA of a modified genome, such as a transgenic plant, are hybridized to solid-supported reference sequences prepared from the source of the suspected non-host DNA in the modified genome. As an example, the modified genome may be that of a transgenic crop plant which has been modified to display, for example, enhanced resistance to disease or herbicides. Such transgenic plants are commonly produced using DNA inserts which were replicated in E. coli, and contamination with undesired E. coli genomic fragments is known to occur. In such a case, the reference library would be one containing these fragments, e.g. a genomic E. coli library.

The particular amounts of probe DNA added to the competitive hybridization reaction can vary depending on the embodiment of the invention. Factors influencing the selection of such amounts include, for example, the structure of probes (single or double stranded), the volume of the hybridization reaction, the quantity of microparticles used, the type of microparticles used, the loading of reference DNA strands on the microparticles, the complexity of the probe DNA, the expected degree of homology between probes and reference sequences, and the like. For example, the amount of probe DNA which would theoretically be required to hybridize to every strand of DNA on a library of microbeads can be estimated from the loading (i.e. the number of reference DNA molecules per bead), the number of beads used, and the average molecular weight of the probe DNA. In practice, this amount is typically multiplied by a factor of about 10-100.

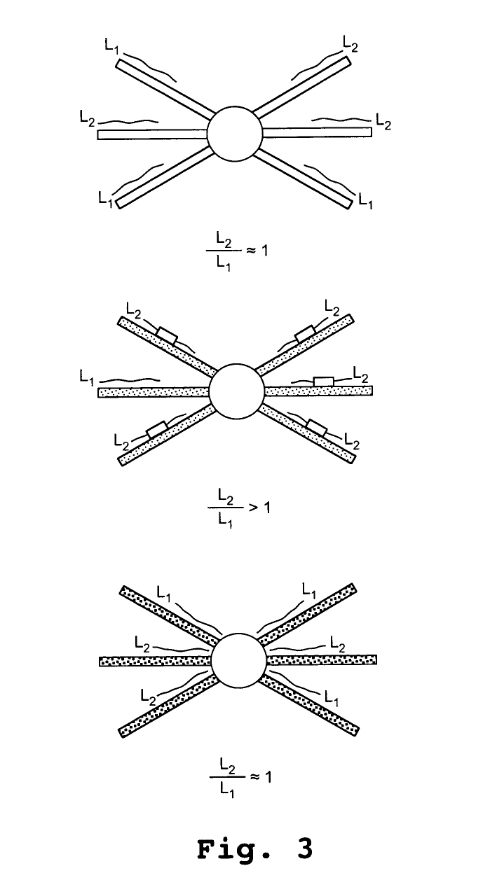

It should be noted that, even though the reference DNA and probe DNA are from different organisms (e.g. from E. coli and from a crop plant), the genomic DNA libraries will frequently have a high enough degree of homology with at least some of the probe sequences that some portion of the probes, even though they do not contain foreign DNA, will hybridize with the reference DNA sequences, as depicted in FIGS. 2-3. For example, in the case of probes from a plant species and reference DNA from E. coli, the ribosomal RNA genes from the different genomes may be nearly identical.

Preferably, the probes are of sufficient length, and hybridization conditions are at the appropriate level of stringency, to permit hybridization between sequences which are homologous but not necessarily identical. For example, in hybridizing probes prepared from soybean DNA with a genomic E. coli reference library (see Examples 4-5), it was found that the optimal probe length was about 120 nucleotides or basepairs or longer (up to about 1.5 kb, the size of the initially sheared fragments). Guidance for selecting suitable hybridization conditions is provided in many references, including Keller and Manak, (cited above); Wetmur, (cited above); Hames et al., editors, Nucleic Acid Hybridization: A Practical Approach (IRL Press, Oxford, 1985); and the like.

A competitive hybridization, in accordance with an embodiment of the invention, is illustrated schematically in FIG. 3. A mixture of probes from the modified genome (labeled L₁, and containing a segment of the foreign DNA) and probes from the unmodified (native) genome (labeled L₂) are competitively hybridized with the reference (foreign) DNA library, whose sequences are cloned on solid phase supports.

Probes from each library which are sufficiently similar to the reference DNA will hybridize to the reference DNA strands on the beads. For example, as noted above, in the case of probe libraries derived from plant species and reference DNA derived from E. coli, the ribosomal RNA genes from the different genomes may be nearly identical. These probes generally represent a small portion of the total probe population, although the proportion can be enhanced by using two rounds of hybridization, as described above and illustrated in FIG. 2.

The majority of genetic material in the two probe libraries, i.e. from the native strain and from the “modified” strain, which is suspected to contain foreign DNA, will be the same. When the “modified” strain does not in fact contain foreign DNA, the two probe libraries will be essentially identical. In this case, in the competitive hybridization, probes from the two different libraries will hybridize to the corresponding reference DNA strands in substantially equal amounts, as shown on the two flanking beads in FIG. 3. Each such bead will contain substantially equal amounts of the two distinct probe labels, which are generally different colored fluorescent dyes. FACS processing of these beads therefore produces a pattern of signals along the diagonal (1:1 ratio of signals).

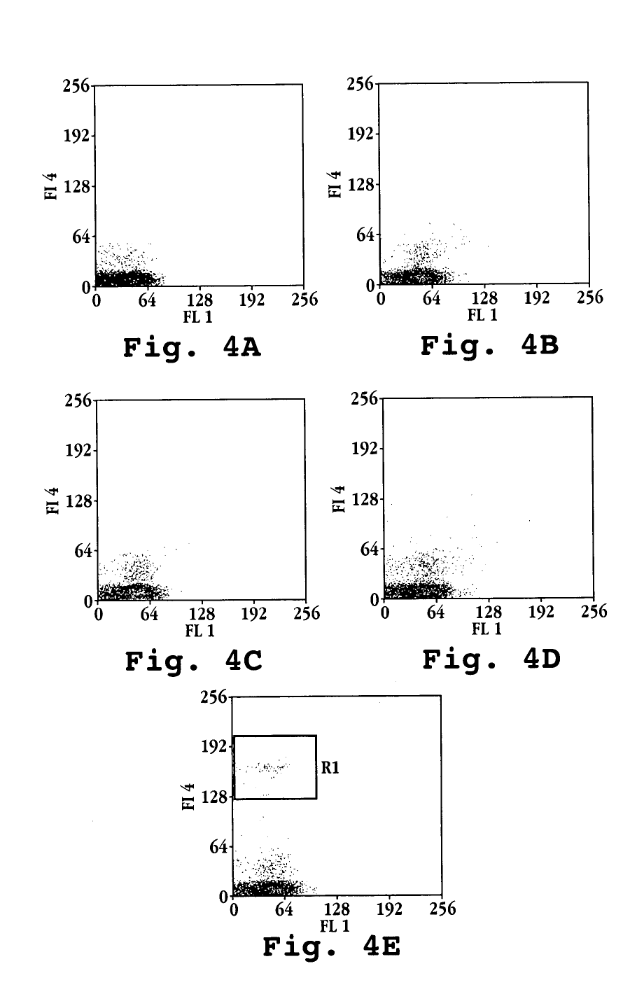

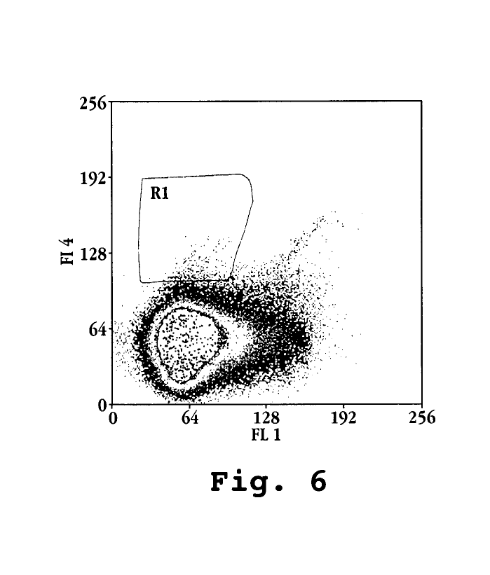

When the modified strain does contain foreign (i.e. reference) DNA, a similar pattern of signals along the diagonal will appear in the FACS output, since the majority of probes in the two populations are still essentially identical. However, a new cluster of signals will appear in an off-diagonal position (see, for example, FIG. 4E or FIG. 6). These signals are due to probes containing the foreign DNA, which hybridize to the corresponding sequences in the reference DNA library to a significantly greater extent than probes from the unmodified genome, as represented by the bead in the center of FIG. 3. Such beads have an unequal ratio of the two different detectable labels, which are generally different colored fluorescent dyes. FACS processing of these beads therefore produces a pattern of signals off of the diagonal (>1:1 ratio of signals), and the beads can thereby be separated from the larger population of beads which appear along the diagonal.

Example 5 describes a competitive hybridization experiment in which beads loaded with reference DNA from a sheared E. coli genomic library (300K beads) were hybridized with GU262 (transgenic genome) probes and with 20 μg PHI1 (non-transgenic genome) probes. To optimize the probe populations, the beads were first hybridized separately with 20 μg GU262 probes and with 20 μg PHI1 probes. PCR was performed to recover the probe strands from the beads. The PCR products were subsequently labeled via another round of PCR, using cy5-T3 as primer for GU262 and fam-T3 for PHI1. The amplified and labeled probes served as probes for competitive hybridization with a similar bead library.

In the procedure described in Example 5, the probes were used in three different ratios: 20:0 μg, 20:20 μg, and 20:60 μg GU262 probes : PHI1 probes. In each case, the beads were analyzed by FACS, to separate beads in which the ratio of labels differed from 1:1 (off-diagonal), showing a preponderance of one type of probe over the other. In repeated experiments, the best results were seen for the third ratio, where generally about 90% of the collected off-diagonal sequences (at gate R1, as shown in FIG. 6) were GU262 probes containing some part of the contaminating E. coli fragment.

VI. Flow Sorting of Microparticles with Unequally Represented Probe Sequences

Microparticles containing fluorescently labeled DNA strands are conveniently classified and sorted by a commercially available FACS instrument, e.g. a FACScalibur (Becton Dickinson). FACS technology is described in such references as Van Dilla et al., Flow Cytometry. Instrumentation and Data Analysis (Academic Press, New York, 1985). For fluorescently labeled DNA strands competitively hybridized to a reference strand, preferably the FACS instrument has multiple fluorescent channel capabilities. Preferably, upon excitation with one or more high intensity light sources, such as a laser, a mercury arc lamp, or the like, each microparticle generates fluorescent signals, usually fluorescence intensities, which are related to the quantity of labeled DNA strands from each sample carried by the microparticle.

Log scaled plots are used in FACS to accommodate the large range of fluorescence signals being measured. It is assumed that DNA probe strands labeled with a single fluorescence dye do not quench each other when hybridized onto the same bead, and that the intensities of two fluorescence signals are linearly proportional to the number of the corresponding probes hybridized onto the beads.

When fluorescent intensities of each microparticle are plotted on a two-dimensional graph, microparticles having equal amounts of probe from the two different sources are on or near the diagonal of the graph. Microparticles having a larger number of probes from one of the sources (generally, the contaminated genome) appear in the off-diagonal regions (see, for Example, FIG. 4E or FIG. 6). Such microparticles can be readily collected by commercial FACS instruments by graphically defining sorting parameters to enclose one or both off-diagonal regions.

VII. Identification of Sorted Genes

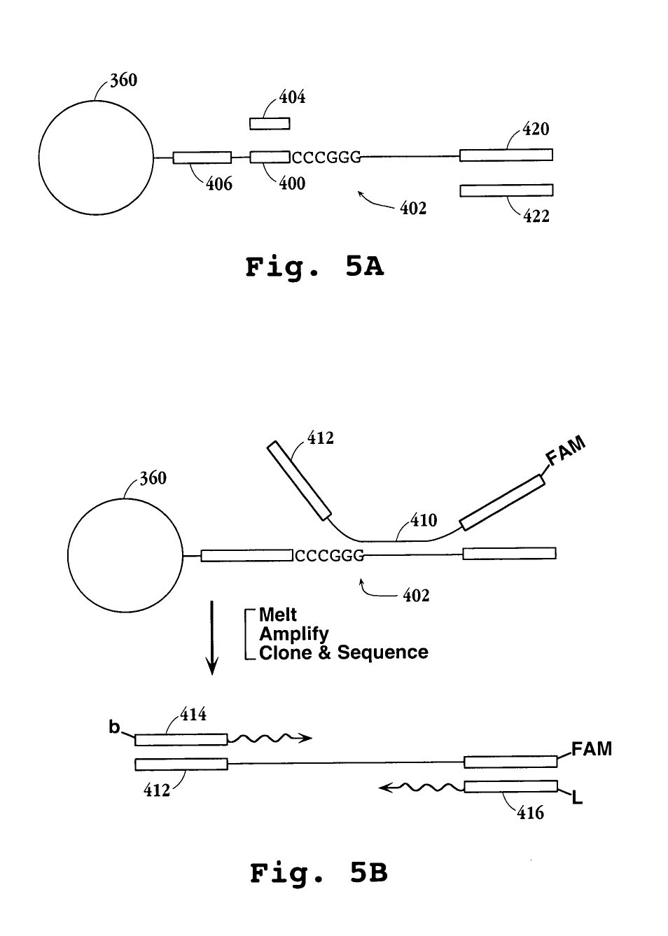

Gene products carried by sorted microparticles may be identified using known DNA sequencing protocols. Suitable templates for such sequencing may be generated in several different ways starting from the sorted microparticles carrying differentially expressed gene products. For example, the reference DNA attached to an isolated microparticle may be used to generate labeled extension products by cycle sequencing, e.g. as taught by Brenner, PCT Pubn. No. WO 96/12039. In this embodiment, primer binding site (400) is engineered into the reference DNA (402) distal to tag complement (406), as shown in FIG. 5A. After isolating a microparticle, e.g. by sorting into separate microtiter wells, or the like, the differentially expressed strands are melted off, primer (404) is added, and a conventional Sanger sequencing reaction is carried out so that labeled extension products are formed. These products are then separated by electrophoresis, or like techniques, for sequence determination. In a similar embodiment, sequencing templates may be produced without sorting individual microparticles. Primer binding sites (400) and (420) may be used to generate templates by PCR using primers (404) and (422). The resulting amplicons containing the templates are then cloned into a conventional sequencing vector, such as M13. After transfection, hosts are plated and individual clones are selected for sequencing.

In another embodiment, illustrated in FIG. 5B, primer binding site (412) may be engineered into the competitively hybridized strands (410). This site need not have a complementary strand in the reference DNA (402). After sorting, competitively hybridized strands (410) are melted off of reference DNA (402) and amplified, e.g. by PCR, using primers (414) and (416), which may be labeled and/or derivatized with biotin for easier manipulation. The melted and amplified strands are then cloned into a conventional sequencing vector, such as M13, which is used to transfect a host which, in turn, is plated. Individual colonies are picked for sequencing.

Although the Examples below employ a model system in which the size and location of the foreign DNA in the modified genome was known, it should be emphasized that the method of the invention does not require advance knowledge of the exact sequences being detected, or where they may appear in the modified genome. The source of the foreign DNA sought to be detected (e.g. a plasmid, expression cassette, replicating organism, infecting organism, or the transformant DNA itself) is used to prepare the reference library, and any sequence in the library can be detected in the modified host genome, using probes prepared from said modified host genome, according to the method of the invention.

VIII. Screening of Multiple Targets

The method of the invention, as described above, can be used for screening a plurality of transgenic organisms, such as a series of transgenic plant lines, for one or more types of foreign DNA. A solid phase supported reference library is prepared, as described, above, for each type of foreign DNA desired to be detected; e.g., each type of foreign DNA that is suspected of being present in any of the series of transgenic organisms. A population of probe sequences is prepared for each different type of unmodified host genome that is represented by the plurality of transgenic organisms; e.g. from the native genome of each type of transgenic plant line represented. A population of probe sequences is likewise prepared for each transgenic organism to be screened.

For each transgenic organism to be screened, its probe population and the probe population obtained from the corresponding native genome, distinguishably labeled as described above, are competitively hybridized with a solid phase supported reference library characteristic of the foreign DNA desired to be detected. Competitive hybridization is carried out in this way for each type of foreign DNA suspected of being present in that transgenic organism. As described above, reference library supports having hybridized thereto a significant preponderance of one type of probe over the other can be detected via the ratio of detectable labels on the supports.

The results of the screening, where the presence or absence of foreign DNA in the transgenic organisms is detected, can then be used as the basis for selecting one or more of the plurality of transgenic organisms according to a predetermined selection criterion. For example, a plant line could be rejected on the basis of unacceptable contamination, or selected on the basis of incorporation of a desired transformant.

### EXAMPLES

The following examples illustrate but are not intended to limit the invention.

### Example 1.

### Preparation of a Probe Library from a Restriction Digest of a pat Expression Cassette Fragment

Suitable probe generation and hybridization conditions for a given assay can be determined by the use of model systems. For example, to determine appropriate probe generation conditions for identification of contaminating E. coli sequences in transgenic soybean line GU262, the 1329-bp ER-ER fragment of pat expression cassette was cloned into pBluscript vector, and the insert was amplified by PCR using M13R/F primers. The PCR product was cut with BamHI, filled in with dGTP, and ligated with T3-ATC adapter (SEQ ID NO: 7,8). PCR was performed with T3 as primer (5′-GCAATTAACCCT CACTAAAGGGAACA-3′ (SEQ ID NO: 7), using the conditions below (Protocol 1). The PCR pattern was evaluated on agarose gel and found to be similar to the original BamHI pattern.

PCR Conditions

1. 94° C. 30 sec

2. 94° C. 5 sec

3. 72° C. 4 min

4. go to 2, 4 more cycles

5. 94° C. 5 sec

6. 70° C. 4 min

7. go to 5, 24 more cycles

8. 72° C. 7 min

9. 4° C.

### Example 2.

### Hybridization of Three Probe Sequences with Corresponding Bead-Supported Sequences

Cy5-labeled probes were generated, as described above in Section IV, for each of three pat cassette BamHI restriction fragments, 315-bp, 485-bp, and 565-bp (referred to respectively as pat-315, pat-485, and pat-565).

Each of these fragments was individually cloned and loaded onto beads, as described, for example, in Section III and illustrated in FIGS. 1A-B. In this case, however, the three bead “libraries” (referred to as “mono bead” libraries) each consisted of only one sequence, for the purpose of determining appropriate hybridization conditions.

A mixture of the three probes was hybridized with each of the corresponding bead mono bead libraries at 65° C. overnight in 4×SSC (saline-sodium citrate buffer: 3M NaCl, 0.3M sodium citrate, pH 7.0): 0.1% SDS (sodium dodecyl sulfate): 25% formamide. The beads were washed with 1×SSC: 0.1% SDS at 65° C. for 30 minutes, followed by 0.1×SSC/0.1% SDS at 65° C. or at 70° C. for 30 minutes (Protocol 2). The probe strands were recovered by T3-primered PCR and analyzed on agarose gel. The results showed that cross hybridization was minimal under these conditions.

### Example 3

### Hybridization of GU262 and PHI1 Probe Libraries with Bead-Supported Reference Sequence (Mono Bead Library)

To prepare the probe populations, GU262 (modified) or PHI1 (parent line) genomic DNA was cut with Sau3A, filled in with dGTP, ligated with T3-ATC adapter, and amplified using T3 as primer, essentially as described above in Section IV.

PCR was then carried out using gene specific PCR primers designed to amplify regions of the modified GU262 genome, which were known for this model system. The four sets of primers, designated pat7/8 for pat cassette, ecol-pat1/2 for the junction of pat cassette and the contaminating E. coli fragment, and ecol3/4 and ecol5/6 for the contaminating E. coli fragment, were chosen for equal representation of the probes. Use of the PCR conditions in Protocol 1, above, gave approximately equal amplification of the four fragments.

The genomic GU262-cy5 probes were hybridized, under conditions given in Protocol 2 above, with the pat-565 “mono bead library” (Example 2). The specificity of the hybridization was evaluated by recovering the probe strand with T3-primered PCR, followed by sequencing of the PCR products. The desired pat sequence was successfully recovered. It was found that raising the wash temperature (to 70° C. from 65° C.) gave better probe specificity but a slightly lower hybridization signal.

### Example 4

### Use of Reduced-Complexity Probe Populations (Two Rounds of Hybridization)

Preparation of “Ecol-pat” mono bead library: Primers designated ecol-7 and ecol-8 were used to amplify the 5514-bp˜6014-bp fragment from E. coli K-12 MG1655 section 310 of 400. The fragment was cut with Sau3A and loaded onto beads, per the procedure described in Section II.

The Ecol-pat “mono bead library” was diluted to 1% with unloaded beads. Labeled probe populations prepared from GU262 or PHI1 DNA (Example 3) were separately hybridized (20 μg each) with the beads, followed by washing, under the conditions given in Protocol 2 above.

The beads were then analyzed by FACS. FACS analyses of control beads, PHI1-hybridized beads, and GU262-hybridized beads are given in FIGS. 4A-C, respectively.

The hybridized probe strands of each population were then recovered from the beads by PCR, and subsequently labeled with another round of PCR. The labeled PCR products were then used as probes for a second round of hybridization. FACS analyses of PHI1-hybridized beads (second round) and GU262-hybridized beads (second round) are given in FIGS. 4D-E, respectively. The beads in gate R1 in FIG. 4E were collected after the 2^{nd} round hybridization, and the probe strands were sequenced. More than 90% of the probes possessed the expected sequence.

### Example 5

### Screening a Transgenic Plant for E. coli Contamination

The transgenic soybean line GU262, known to contain not only the transgenic marker pat expression cassette, but also a 4.2 kb E. coli genomic DNA fragment, was used for screening. Using the method of the invention, 2.9-kb E. coli fragments were detected in the 10⁹-bp genome of the transgenic plant, as follows.

A sheared E. coli genomic bead bed was used as the reference library. E. coli genomic DNA was sheared to fragments having an average size of 1.5-2 kb. The fragments were cloned into tag vectors and loaded onto beads, as described above in section III and illustrated in FIGS. 1A-B.

Genomic DNA from GU262 and its isogenic parent line PHI1 were used to prepare probe libraries, as described above, with fragments produced by restriction with Sau3A, RsaI, or Taq I, or by shearing to an average of 2 kb, followed by reduction to about 600 bp by Bal31 treatment.

The sheared E. coli genomic reference library, loaded on microparticles (300K), was hybridized with 20 μg GU262 and PHI1 probes (separately), under conditions given in Protocol 2, and PCR was performed to recover the probe strands from the beads. The PCR products were subsequently labeled via another round of PCR, using cy5-T3 as primer for GU262 and fam-T3 for PHI1. The amplified and labeled probes served as probes for the second round of (competitive) hybridization. The probes were combined in three different ratios for the competitive hybridization: 20 μg:0 μg, 20 μg:20 μg, and 20μg:60μg GU262:PHI1.

FACS analysis for the 20:60 ratio of GU262 to PHI1 is shown in FIG. 6. (Note that under such conditions, where a high concentration of PHI1 probes is used, some quantity of FAM-labeled PHI1 probes are expected to hybridize to other solution-phase probes, rather than to the microparticle-bound E. coli sequences. Accordingly, the excess of FAM labeled probes does not necessarily appear in the FACS sorting process.) The beads in gate R1 (FIG. 6) were collected after hybridization, and the probe strands were examined by sequencing. In repeated experiments, approximately 90% of these sequences were from the contaminating E. coli fragment. The identified sequences encompassed about 3.4 kb, for probes prepared by restriction, and about 3.6 kb for probes prepared by shearing and restriction.

While the invention has been described with reference to specific methods and embodiments, it will be appreciated that various modifications may be made without departing from the invention.

It is claimed:

1. A method for detecting foreign DNA in a modified host genome, suspected to contain said foreign DNA, which is not present in the unmodified host genome, the method comprising the steps of:
(a) competitively hybridizing first and second populations of polynucleotide probes with a reference DNA population, said reference DNA population comprising DNA sequences characteristic of said foreign DNA, wherein different DNA sequences are attached to separate solid phase supports in clonal subpopulations;
wherein each of said first population of polynucleotide probes comprises a DNA fragment from the unmodified host genome, and has a first label; and each of said second population of polynucleotide probes comprises a DNA fragment from the modified host genome, and has a second, distinguishable label;
thereby forming duplexes between the DNA sequences of the reference DNA population and the polynucleotide probes;
(b) sorting the solid phase supports, according to the ratio of said first label to said second label on the duplexed probes hybridized to each support;
(c) selecting solid phase supports having a ratio of fluorescent signals which falls within a selected range of values different from 1:1; and
(d) identifying the attached sequences or hybridized probes on the selected solid phase supports.

2. The method of claim 1, wherein the first and second labels, respectively, are first and second distinguishable fluorescent labels.

3. The method of claim 1, wherein said solid phase supports are microparticles, and said microparticles are sorted by FACS, according to the ratio of fluorescent signals generated by said fluorescent labels on each microparticle.

4. The method of claim 1, wherein said identifying comprises sequencing at least a portion of said hybridized probes.

5. The method of claim 1, wherein said probe populations are prepared by:
preparing a restriction digest or sheared digest of fragments from said native genome or said modified genome;
ligating pairs of PCR primers to said fragments, wherein each said pair of PCR primers includes at least one labeled primer; and
amplifying said fragments by PCR.

6. The method of claim 1, wherein said probes have a length such that some portion of the first population of probes are able to hybridize with sequences of the reference DNA population, under the conditions of said hybridizing.

7. The method of claim 1, further comprising, prior to step d):
separately hybridizing each said probe population with the reference DNA population;
recovering each said probe population from the solid phase supports; and
amplifying each recovered probe population by PCR, using primers labeled with said first or second labels, respectively.

8. The method of claim 1, further comprising the steps, prior to said competitive hybridization, of:
(i) providing said reference DNA population, comprising DNA sequences characteristic of said foreign DNA, wherein different sequences are attached to separate solid phase supports in clonal subpopulations;
(i) providing said first population of polynucleotide probes, each comprising a DNA fragment from the unmodified host genome, not containing said foreign DNA, and having a first label;
(iii) providing said second population of polynucleotide probes, each comprising a DNA fragment from the modified host genome, suspected to contain said foreign DNA, and having a second, distinguishable label.

9. The method of claim 8, wherein said modified host genome is one of a plurality of transgenic organisms, and the method comprises:
carrying out step (i) for each type of foreign DNA suspected of being present in said plurality of transgenic organisms;
carrying out step (ii) for each different type of unmodified host genome represented in the plurality;
carrying out step (iii) for each transgenic organism of the plurality;
carrying out steps (a)-(d) for each transgenic organism of the plurality, and for each type of foreign DNA suspected of being present in that organism, to determine the presence or absence of foreign DNA in each organism of the plurality; and
selecting one or more of the plurality of transgenic organisms, according to a predetermined selection criterion based on the presence of the foreign DNA in the organism.

10. The method of claim 8, wherein said transgenic organisms are transgenic plant lines.

11. A kit for use in detecting foreign DNA in a modified host genome, by competitive hybridization of probes to a reference library, the kit comprising:
(i) a reference nucleic acid library containing DNA sequences characteristic of said foreign DNA, wherein different sequences are attached to separate solid phase supports in clonal subpopulations;
(ii) a first plurality of probes, derived from a nucleic acid library from the unmodified host genome, not containing said foreign DNA, and having a first label, and
(iii) a second plurality of probes, derived from a nucleic acid library from the modified host genome, suspected of containing said foreign DNA, and having a second label distinguishable from said first label.

12. The kit of claim 11, wherein said probes have a length such that some portion of the first population of probes are able to hybridize with sequences of the reference DNA population under conditions of said hybridization.
